(19) United States
(12) Patent Application Publication
(10) Pub. No.: US 2017/0364653 A1
(43) Pub. Date: Dec. 21, 2017

(54) MEDICAL DATA EXTRACTION AND MANAGEMENT FOR EFFICIENT, SECURE SUPPORT OF VARIOUS INFORMATION SYSTEMS

(71) Applicant: Neximatic, Inc., Chicago, IL (US)

(72) Inventors: Bobby Wong, Chicago, IL (US); Jack Li, Newton, MA (US)

(73) Assignee: Neximatic, Inc., Chicago, IL (US)

(21) Appl. No.: 15/535,525

(22) PCT Filed: Dec. 18, 2015
(86) PCT No.: PCT/US15/66661

§ 371 Date: Jun. 13, 2017

Related U.S. Application Data
(60) Provisional application No. 62/094,352, filed on Dec. 19, 2014.

(51) Int. Cl. G06F 19/00; G06F 17/30; G06F 21/62

(52) U.S. Cl. CPC G06F 19/3418; G06F 21/6245; G06F 17/30867; G06F 19/322; G06F 19/327

(57) ABSTRACT

At a first computer connected to a medical monitoring device, first data are received from the monitoring device repetitively. The first computer is within a first local network, the monitoring device is configured to monitor characteristics of a patient, and the first data represent monitored characteristic(s) of the patient or associated metadata. The first data are sent from the first computer to a second computer via the local area network and a public network. The second computer is outside the first local area network and connected to the public network, and the first data is sent from the first computer to the second computer based on an IP address stored at the first computer. A value of one of the monitored characteristics is determined as within a predetermined range, and a polling frequency for each of the monitored characteristics is set to a common frequency based on that determination.

### CROSS-REFERENCE TO RELATED APPLICATIONS

This application claims priority from co-pending U.S. Provisional Application Ser. No. 62/094,352 filed Dec. 19, 2014, the entirety of which is hereby incorporated by reference herein.

### BACKGROUND

The present disclosure is generally directed to efficient, secure, and scalable techniques for querying, retrieving and managing various medical and health related data. More specifically, the present disclosure is directed to the use of improved medical device connectivity solutions in clinical settings to support different requirements from various information systems simultaneously.

As medical information systems such as electronic patient record (EPR) systems proliferate, clinical staff can enter their medical observations, diagnoses and orders into the systems. Systems called medical device connectivity solutions query and retrieve medical data (e.g., blood pressure, heart rate, blood oxygen level, etc.) measured by medical instrumentation devices and provide the medical data to medical information systems. Medical device connectivity solutions traditionally have been facility-centric in the sense that a medical device connectivity solution in a medical facility (e.g., hospital) utilizes the facility's network to provide data to a medical information system within the same facility network under the same ownership as the medical device connectivity solution. Such a facility-centric medical device connectivity solution has numerous drawbacks, including limited scalability, reliability and upgradability. Moreover, such a facility-centric solution does not provide access to the medical data outside of the facility.

A traditional facility-centric medical device connectivity solution requires a large number of the facility's network configurations to be stored in a computer memory, making such an approach not scalable in a large facility. Referring to FIG. 1, a hospital network typically comprises many smaller networks (e.g., local area networks) 110a, 110b, 110c interconnected by one or more gateways 111. A firewall 130 connected to gateway 111 governs communications with an external network 140 (e.g., a public network such as the Internet). Often, a medical monitoring device 102 connected to a medical device connectivity solution 104 is configured in one network 110a (e.g., corresponding to the first floor of a hospital), and medical information systems 112a, 112b are configured in topologically distant networks 110b, 110c (e.g., corresponding to the eighth and ninth floors, respectively, of the hospital). Information systems 112a and 112b and other information systems described herein may be any computer systems that seek to obtain data or records regarding a patient or treatment of a patient. Various computing devices, e.g., notebook computer 124a, tablet computer 124b, and desktop computers 124c, 124d, 124e, may be connected to respective local networks. These different networks often govern access by using different security protocols and different administration settings, requiring a large amount of network configuration data to be stored in medical device connectivity solution 104 in order to provide services to information systems 112a,112b.

Medical device connectivity solution 104 needs to store network configuration data (e.g., IP address data) that enables access to local network 110a on the first floor and also needs to store network configuration data (e.g., IP address data) of information systems 112a and 112b. In some situations, a hospital's information technology (IT) staff may configure the communication between medical device connectivity solution 104 and medical information systems 112a and 112b in a Virtual Local Area Network (VLAN). A VLAN segregates and prioritizes its traffic (e.g., paths 120a and 120b) from other network traffic (e.g., paths 122a, 122b, 122c) to improve latency of communication. In the example shown in FIG. 1, medical device connectivity solution 104 has to store additional settings that enable VLAN communications. As network complexity and size within a medical facility increase, the number of network configurations also increases, making a facility-centric solution particularly difficult to scale.

In addition to scaling-related challenges, the large number of network configurations that must be stored in these typical medical device connectivity solutions reduces their operational reliability. Networking equipment typically has a short upgrade cycle. As network equipment is upgraded, the associated networking configurations typically change. Maintaining a large number of frequently changing networking configurations introduces a high degree of risk in terms of operational reliability and security.

The software in traditional medical device connectivity solutions needs to be maintained regularly by the manufacturers for bug fixes or updates for new features. These medical device connectivity solutions are typically installed at locations where physically performing updates on-site is difficult, e.g., on a wall-mount of a patient monitor or on a pole of a patient monitor's wheeler. Many traditional medical device connectivity solutions require a facility's IT staff to interact physically with the medical device connectivity solutions for maintenance. There are two common ways to do this: (a) the facility's IT staff have to download software updates from the manufacturers' websites and direct the updates manually to the medical device connectivity solutions on-site, or (b) the facility's IT staff have to physically start a webcast on the medical device connectivity solutions on-site and give remote access to the manufacturers' personnel so that the manufacturers' personnel can direct the updates remotely.

Some prior art medical device connectivity solutions try to reduce the amount of interaction on the part of the facility's IT staff by installing a Remote Desktop program or similar program on the medical device connectivity solutions for support of a remote update procedure. These programs require a server computer 105 to be installed and running on each medical device connectivity solution 104, as shown in FIG. 2. Server 105 opens a port 106 to listen for any incoming client request. This open port 106 (e.g., port number 3389 for Remote Desktop protocol) has to be registered by firewall 130 of the medical facility in order for any external request from a medical equipment manufacturer's computer 150 to pass through the firewall 130 (e.g., via port 131) to port 106 of the server 105 (this path is labeled 152). This registration requirement adds an additional network configuration burden to medical device connectivity solution 104. The manufacturer's staff traditionally initiate such requests remotely. The requests pass through the firewall 130 and arrive at the open port 106 of server 105 to establish connections. After establishing connections, the manufacturer's personnel then have access to the medical device connectivity solutions and can direct updates. The foregoing traditional remote update procedure has a dangerous security vulnerability. If a facility's IT staff inadvertently provides a credential to a person with a malicious intent, this person could impersonate someone else and connect to medical device connectivity solution 104.

Traditional medical device connectivity solutions are facility-centric and can only support medical information systems of the same ownership located within a particular facility's networks. However, there are many situations where medical data are needed by medical information systems that are located outside of the facility network and/or of different ownership. For instance, suppose an independent physician group of anesthesiologists is contracted by a hospital to provide anesthesia services to the hospital's patients during surgical operations. A facility-centric medical device connectivity solution provides medical data regarding the surgical services within the network of the facility. However, these anesthesiologists may want to record the medical data regarding the surgical services in their own information system located outside of the hospital's network (e.g., separated from the hospital's network by another network such as the Internet 140), and a traditional facility-centric medical device connectivity solution lacks such a capability, as shown in FIG. 3. In FIG. 3, medical information system 310 of the group of anesthesiologists is outside the networks of the medical facility containing medical device connectivity solution 104 and medical information systems 112a and 112b.

The different medical information systems of different ownership that access the medical data provided by medical device connectivity solution 104 may have different purposes. For instance, an information system such as an electronic patient record (EPR) system queries and retrieves continuous medical data during a medical procedure for a record-keeping purpose. Another information system, e.g., a medical procedure review information system, queries and retrieves other kinds of data, such as duration of procedures or medical events during procedures, in additional to the medical data, in order to review the performed procedures collectively. Traditionally, medical device connectivity solutions do not manage the distribution of information and do not have any capability to monitor or enforce different privileges and access rights with respect to the details regarding purposes (e.g., how often queries can be performed, what kind of data can be retrieved, the time frame of the data that can be retrieved) and ownership.

Furthermore, information systems can be implemented using various types of computing devices (e.g., a server, a laptop computer, a tablet computer, a smart phone or a wearable computer) and thus may have varying amounts of processing power and memory and different application needs. For example, a telemedicine platform running on a computer may need to display medical data from multiple rooms for 2-10 hours. In contrast, a vital sign charting application running on a tablet computer may focus on one room for the most recent hour of medical data. These information systems can run simultaneously. It is not practical to provide to these various information systems the same set of data obtained from medical monitoring device 102 without customization (e.g., condensation or summarization of data, or a specific granularity or subset of data) to the different needs. Indeed, for many medical information systems it is desirable not to provide much data, in order to conserve resources. The traditional approach shown in FIGS. 1-3 for using medical device connectivity solution 104 is inflexible to support the different requirements of various information systems 112a, 112b, 310 and may provide those information systems with too much data or data in a different format, time interval, or granularity than desired.

Another constraint of traditional medical device connectivity solutions is that they are limited to providing medical data (e.g., measurable physiological data such as blood pressure, heart rate, oxygen saturation level) and do not have the ability to provide other types of data such as information about a medical instrument (e.g., usage, downtime, present operational status).

### SUMMARY

In some embodiments, a system includes a first computer located in a patient care facility, with the first computer connected to a first local area network, and a set of one or more computers outside the first local area network. The first computer includes a first processor, a memory, a first communications interface connecting the first computer to one or more medical monitoring devices configured to monitor a plurality of characteristics of a patient in the patient care facility, and a second communications interface connecting the first computer to the first local area network. The first computer further includes a non-transitory computer readable storage medium tangibly embodying first instructions executable to cause the first processor to perform various operations, including: receive first data from the one or more medical monitoring devices, the first data representing the plurality of monitored characteristics of the patient or metadata associated with the plurality of monitored characteristics; send the first data via the second communications interface to a predetermined IP address, wherein the predetermined IP address is stored in the memory of the first computer; detect that a value of one of the monitored characteristics is within a predetermined range; and set a polling frequency for each of the monitored characteristics to a common frequency based on said value being within the predetermined range.

The set of one or more computers includes a second processor, a database, a third communications interface connecting each computer in the set to a public network, wherein the first local area network is connected to the public network, and a non-transitory computer readable storage medium tangibly embodying second instructions executable to cause the second processor to perform various operations including: receive the first data via the third communications interface; store the first data in the database; receive a data request from a medical information system via the public network, wherein the medical information system is registered with one of the computers in the set of one or more computers; verify a registration status of the medical information system; authenticate the medical information system; and only if the registration status is successfully verified and the medical information system is successfully authenticated, send second data to the medical information system, the second data representing at least one monitored characteristic of the patient, usage data associated with the medical monitoring device, or status data associated with the medical monitoring device, or a combination thereof.

In some embodiments, a method comprises receiving, at a first computer connected to one or more medical monitoring devices, first data from one of the medical monitoring device repetitively according to a programmable time interval, wherein the first computer is within a first local area network, the one or more medical monitoring devices are configured to monitor a plurality of characteristics of a patient in a patient care facility, and the first data represent one or more monitored characteristics of the patient or metadata associated with said one or more monitored characteristics. The method further comprises sending the first data from the first computer to a second computer via the local area network and a public network, wherein the second computer is outside the first local area network and is connected to the public network, and the first data is sent from the first computer to the second computer based on an IP address of the second computer stored in a memory of the first computer. The method further comprises determining that a value of one of the monitored characteristics is within a predetermined range, and setting a polling frequency for each of the monitored characteristics to a common frequency based on said value being within the predetermined range.

In some embodiments, a method comprises receiving first data sent from a first computer connected to a medical monitoring device, wherein the first computer is within a first local area network, the medical monitoring device is configured to monitor at least one characteristic of a patient in a patient care facility, the first data represent one or more monitored characteristics of a patient or metadata associated with said one or more monitored characteristics. A second computer outside the first local area network receives the first data. The first data are stored in a database. The method further comprises receiving a communication request from a medical information system via a second local area network and the public network, wherein the medical information system is on the second local area network and is registered with the second computer. The method further comprises verifying a registration status of the medical information system and authenticating the medical information system using an authentication protocol. If the registration status is successfully verified and the medical information system is successfully authenticated, a portion of the first data is sent to the medical information system via the public network and the second local area network.

### BRIEF DESCRIPTION OF THE DRAWINGS

The following will be apparent from elements of the figures, which are provided for illustrative purposes and are not necessarily to scale.

FIG. 1 is a block diagram of a system including a traditional facility-centric medical device connectivity solution.

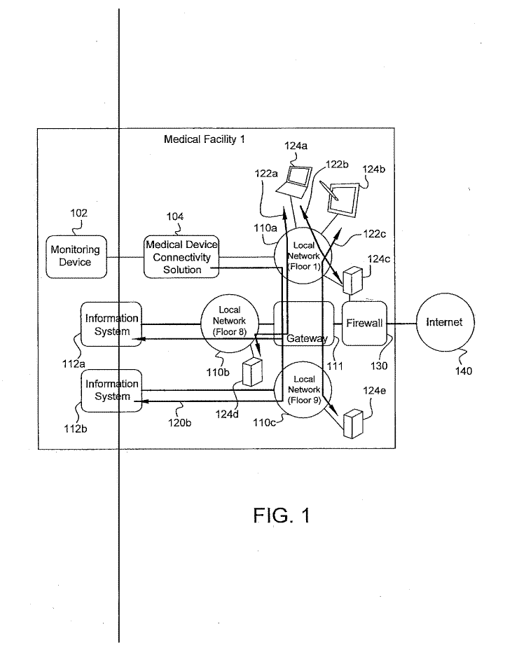

FIG. 2 is a block diagram illustrating a traditional remote update procedure.

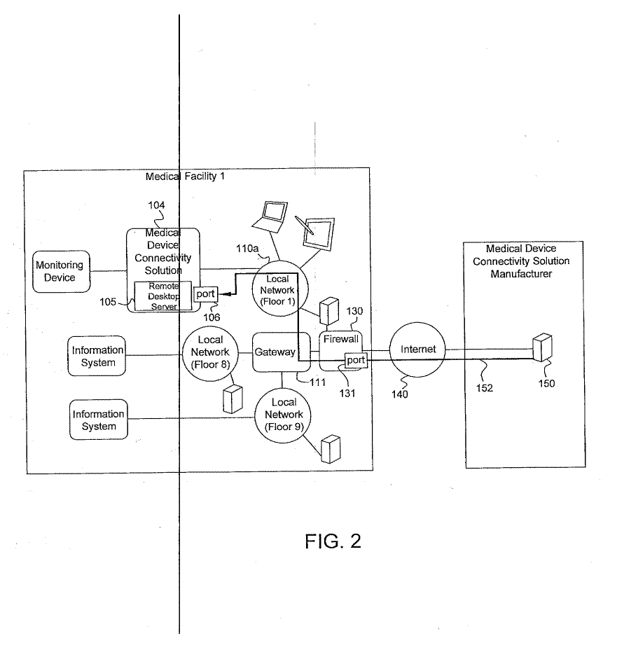

FIG. 3 is a block diagram illustrating the difficulty of transporting data outside of a facility when a traditional facility-centric medical device connectivity solution is used.

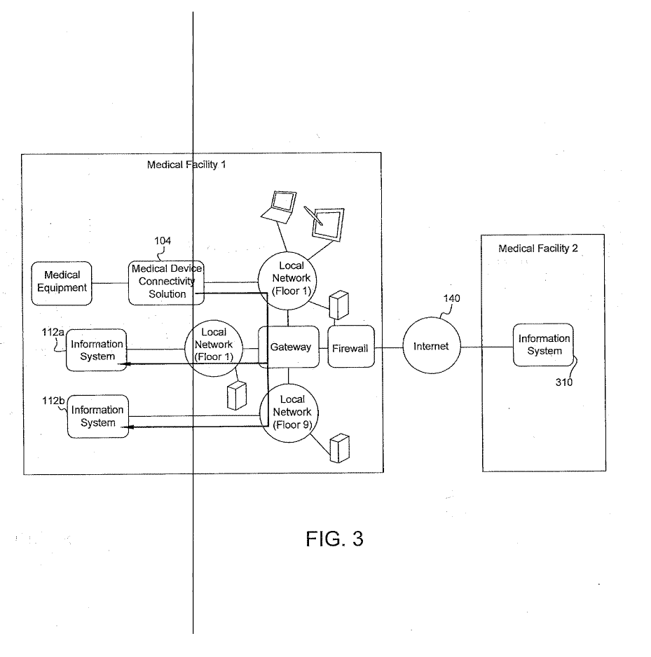

FIG. 4 is a diagram of a system including a data processor, data repository, and system-wide manager in accordance with some embodiments of the present disclosure.

FIG. 5 is a block diagram of a data processor in accordance with some embodiments.

FIG. 6 shows example functionality of a data processor in accordance with some embodiments.

FIG. 7 shows example functionality of a data repository in accordance with some embodiments.

FIG. 8 shows communication between a data processor, data repository, and system-wide manager in accordance with some embodiments.

FIG. 9 is a block diagram of a computer architecture that may be used for implementing a data repository and/or system-wide manager in accordance with some embodiments.

### DETAILED DESCRIPTION

This description of the exemplary embodiments is intended to be read in connection with the accompanying drawings, which are to be considered part of the entire written description.

Various embodiments of the present disclosure address the foregoing challenges and other challenges associated with traditional facility-centric medical device connectivity solutions. In some embodiments, an Internet-supporting or cloud-based (as opposed to facility-centric) medical data streaming solution (connectivity solution) improves scalability, reliability and upgradability and provides the capability to manage medical data distribution to different information systems within and outside of a medical facility.

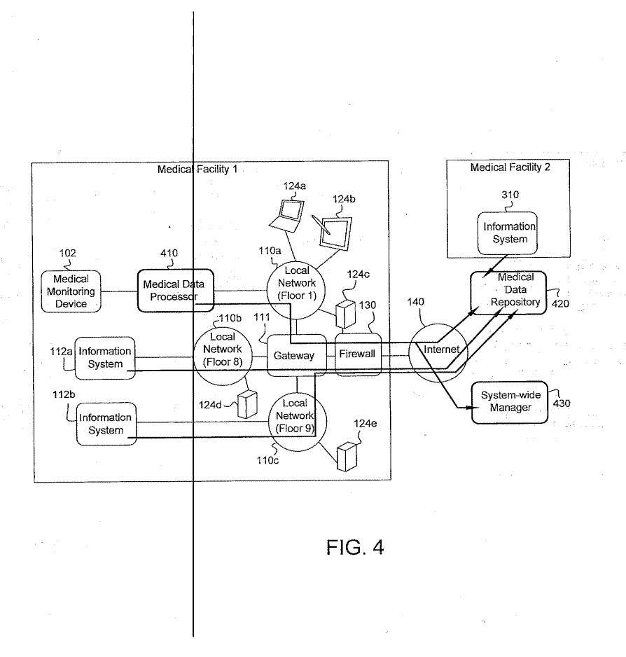

FIG. 4 is a diagram of a system including a data processor 410, data repository 420, and system-wide manager 430 in accordance with some embodiments of the present disclosure. A computer referred to as medical data processor 410 is located in a patient care facility (e.g., a medical facility such as a hospital or clinic) and is connected to medical monitoring device 102. Data processor 410 may also be referred to as a data extractor. Although a single data processor 410 is shown in FIG. 4 for graphical convenience and “data processor 410” (in the singular) may be described below, multiple data processors 410 may be used in some embodiments, and discussion of data processor 410 is also applicable to multiple data processors 410.

Data processor 410 is configured within local network 110a, which is connected to gateway 111. Firewall 130 governs traffic between gateway 111 and an external network 140 which may be a public network such as the Internet. A medical data repository 420 and a system-wide manager 430 are connected to network 140. Medical data repository 420 and system-wide manager 430 may be collocated (e.g., within a single physical computer) or may be physically separated. Thus, medical data repository 420 and system-wide manager 430 may be implemented using one or more computers. Functionalities pertaining to medical data repository 420 and system-wide manager 430 are described below, and one of ordinary skill in the art understands that such functionalities may be distributed across a set of one or more computers. Information system 310 of a medical facility other than the one containing data processor 410 is connected to medical data repository 420.

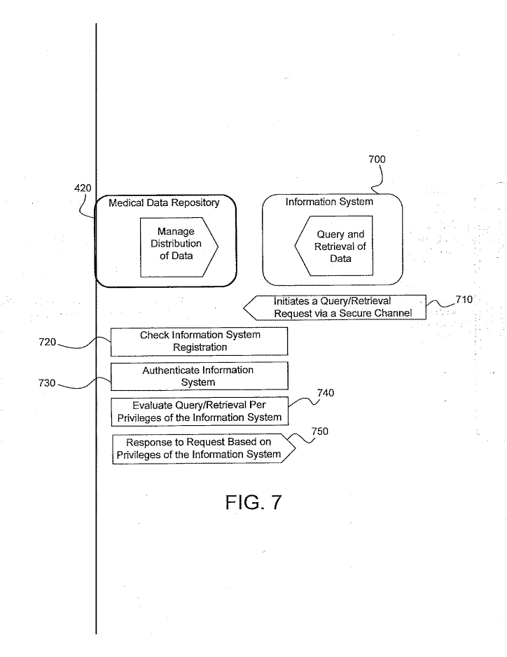

Medical data repository 420, which can be located on-customer-premise or off-customer-premise, or distributed between the two, including the use of cloud architectures, may be configured to receive data from multiple data processors 410, store the data, and manage the distribution of the data to consumers of such data (e.g., medical information systems). In some embodiments, only registered and authenticated data processors 410 can communicate with data repository 420 (e.g., to store data at a database of repository 420) and with system-wide manager 430, and only registered and authenticated information systems can query and retrieve data from data repository 420. Registration and authentication are described in more detail further below in the context of FIG. 7. Data repository 420 may include one or more mass storage devices for storing data, e.g., magnetic, magneto optical disks, or optical disks. Data repository 420 does not have to be a single device and may include a distributed storage architecture.

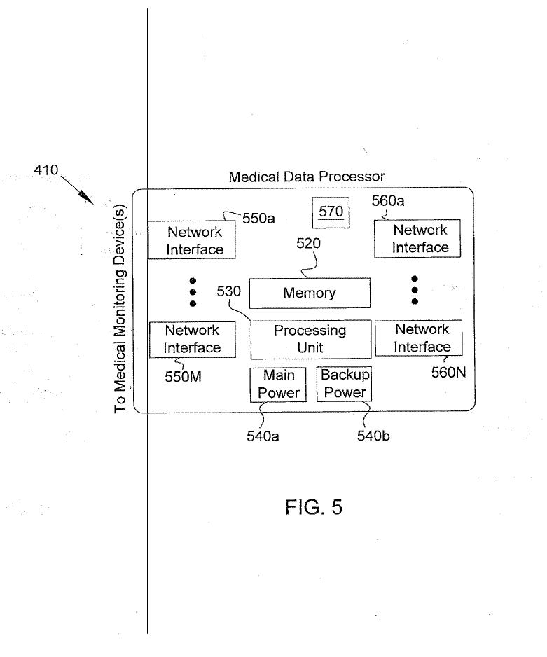

FIG. 5 is a block diagram of medical data processor 410 in accordance with some embodiments. Data processor 410 includes a memory 520, a processing unit (e.g., microprocessor) 530, and one or more power supply units 540a, 540b (e.g., main power and backup power). A first communications interface is used for connecting with one or more medical monitoring devices 102 wirelessly or via a wired connection. The first communications interface may include one or more network interface ports 550-1, . . . , 550-M (M being a positive integer) configured to monitor a plurality of characteristics of a patient in the patient care facility. A second communications interface is used for connecting to local network 110a wirelessly or via a wired connection. The second communications interface may include one or more network interface ports 560-1, . . . , 560-N (N being a positive integer).

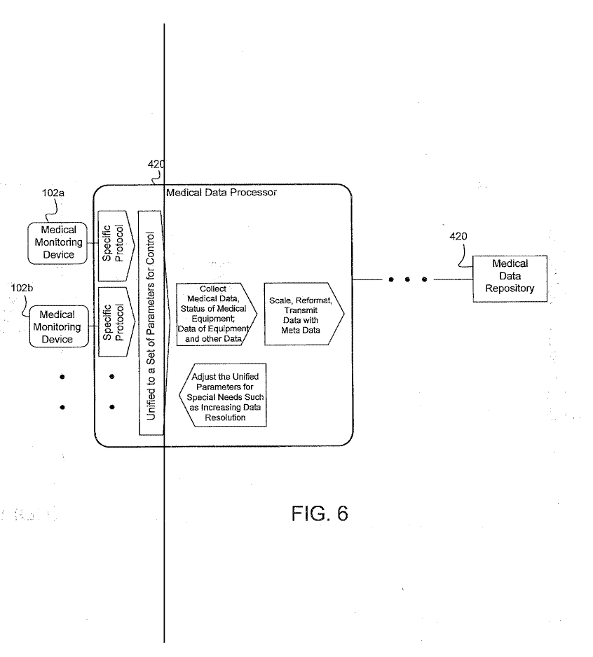

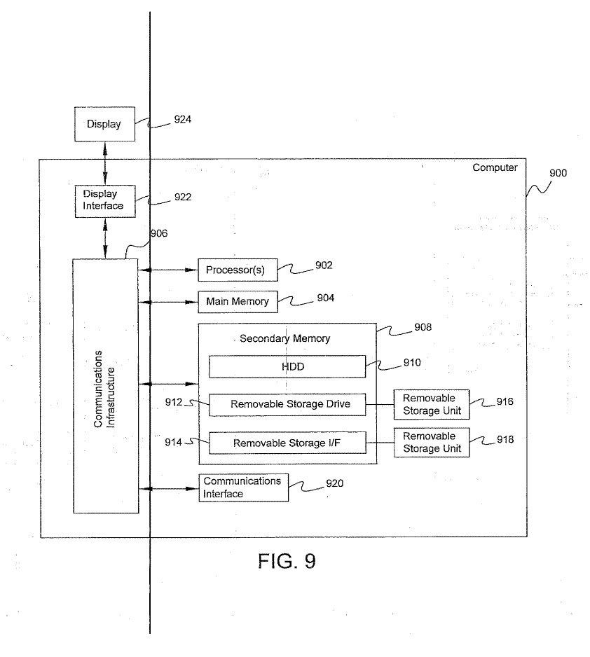

A non-transitory computer readable storage medium 570, which may be part of or separate from memory 520, tangibly embodies a set of instructions that are executable by processor 530. When executed, the instructions cause processor 530 of data processor 410 to receive data from the one or more medical monitoring devices (shown as 102a, 102b, . . . in FIG. 6; these may referred to collectively as medical monitoring devices 102) representing monitored characteristics (e.g., heart rate, blood pressure, etc.) of a patient or metadata associated with the monitored characteristics (e.g., time or location of measurement, type of monitoring device, type or parameters associated with the measurement, etc.). The data may be retrieved from monitoring devices 102 on a regular basis (e.g., every second, minute, hour, etc.), scaled (e.g., between respective measurement units), reformatted (e.g., per varying equipment manufacturer data formats or a standardized data format), and sent to medical data repository 420 as shown in FIG. 6. In some embodiments, the IP address of data repository 420 is stored in memory 520 and is used for sending data to data repository 420. Medical data repository 420, which is connected via a communications interface to public network 140 (e.g., the Internet), receives the data sent by data processor 410 and stores the data in a database of data repository 420, which may be implemented using any of the memory or storage components shown in FIG. 9, for example.

Later, data repository 420 receives a data request from a medical information system (e.g., any of information systems 112a, 112b, 310) registered with data repository 420. The data request is received via public network 140. Such a medical information system is a downstream consumer of data, and additional details regarding communication between the medical information system and data repository 420 are described below in the context of FIG. 7.

In addition to medical data or metadata associated with such medical data, the data received by data processor 410 from medical monitoring device 102 may include usage data (e.g., usage logs or other data showing past usage of the monitoring device) and/or data regarding the operational status of medical monitoring device 102 (e.g., whether it is powered on or off, operating normally or abnormally, in an error state, or one or more status conditions regarding operation). Processor 530 may send any of these types of data via the second communications interface to system-wide manager 430. In some embodiments, the IP address of system-wide manager 430 is stored in memory 520 and is used for sending data to system-wide manager 430.

Various medical equipment manufacturer may have different protocols for data access regarding its equipment. Some protocols may push data out (e.g., periodically) whereas others may require a query (pull). As shown in FIG. 6, data processor 410 may use each specific protocol for accessing respective monitoring devices 102a, 102b, etc., and data processor 410 presents a unified interface to those monitoring devices from the perspective of downstream data consumers, e.g., a unified set of parameters for data access. This enables the frequency of polling for any individual measurement to be controlled by logic associated with various conditions. For example, if one measurement from one monitoring device 102a enters a range associated with an emergency (e.g., pulse below a predetermined threshold such as 60 beats per minute), data processor 410 can increase the polling frequency for all measurements from all the monitoring devices 102a, 102b, etc., connected to data processor 410. In some embodiments, in this emergency scenario the polling frequency for all monitored characteristics is increased, e.g., to a common polling frequency, and in other embodiments the polling frequency for one or more monitored characteristics is increased but they are not necessarily all increased to a common polling frequency. When the emergency passes (e.g., a measurement has a value outside a predetermined range, such as the pulse rising above 60 beats per minute), the polling frequency for one, more than one, or all monitored characteristics may be decreased , e.g., to their former settings prior to the emergency. In this way, data may be captured and transmitted to data repository 420 at a fine granularity during emergencies, which promotes optimal patient care, and data may be captured and transmitted at a coarse granularity at other times, which conserves system resources and improves network performance.

Data processor 410 may have a small form factor so that it is portable and/or mountable to stationary and/or mobile medical equipment. Data processor 410 may be implemented in the form of any type of computing device, e.g., a smartphone, tablet computer, desktop computer, embedded computing device, or other computing device. In some embodiments, multiple data processors 410 may be connected directly or indirectly to a single piece of medical equipment (e.g., heart rate monitor). The multiple data processors 410 may coordinate amongst themselves, e.g., to share information, and such coordination may be achieved via wired communication or wireless communication (e.g., Bluetooth, RF data transmission, or other any other wireless communication technique). Multiple data processors 410 may be nodes in a communication network sharing storage resources. In one embodiment, one of the data processors 410 in a patient room may collect data from one, more than one, or all the other data processors in the patient room (which may themselves be collecting data from respective pieces of medical equipment) and send the collected data to data repository 420. In this way, network communications load may be reduced.

In some embodiments, one or more data processors 410 may be dynamically assigned or re-assigned to different pieces of medical equipment on an as-needed basis, and respective data processors 410 may service different numbers of pieces of medical equipment. In an embodiment including an intelligent network of data processors 410, the data processors 410 may include contention resolution algorithms to transmit data via a local network 110a at opportune times.

In some embodiments, only one network configuration is stored in memory 520 to enable data from respective medical monitoring devices 102 to be transported. This one network configuration may be network configuration data of local network 110a which is connected to (has a path or route to to) network 140. Such embodiments requiring storage of only one network configuration yield increased reliability and ease of maintenance and decreased resource requirements in terms of storage at data processor 410.

Data processor 410 may retrieve and store configuration data regarding the medical equipment 102 from which data is to be extracted or has been extracted, regarding local network 110a, and regarding its own operation (e.g., downtime or usage statistics for data processor 410). Data processor 410 may be configured to monitor the status of the medical equipment 102 (such as on/off state of the medical equipment), of the local network 110a and of its own health (such as AC power on/off status). Data processor 410 may regularly communicate, in a secured manner via networking interface(s) 560-1, . . . , 560-N, configuration/status data pertaining to medical equipment 102, data processor 410 or local network 110a to system-wide manager 430, which may be located on-customer-premises or off-customer-premises. When there is an issue (e.g., a non-functional medical monitoring device 102, an error condition at data processor 104, or an outage or slowdown on local network 110a), system-wide manager 430 may generate and send an alert, e.g., to an IT unit for corrective action before the issue escalates to a more serious problem.

In some embodiments, in order to establish a connection to data repository 420 and system-wide manager 430, data processor 410 only needs an association to a local network (e.g., local network 110a) that has a route to the Internet 140, thereby reducing the networking configuration storage/maintenance/management burden compared to traditional approaches.

In some embodiments, data processor 410 detects an anomalous connection between one of the medical monitoring devices 102a, 102b, etc. and data processor 410 and sends to data repository 420 or system-wide manager 430 a notification of the anomalous connection. For example, if the connection between data processor 410 and one of the medical monitoring devices is a wired connection, data processor 410 may first test the wired interface. Then, if the wired interface is OK, data processor 410 may send a request for data to the medical monitoring device and check for a response to the request from the medical monitoring device. If there is no response or a faulty response, then the connection may be deemed to be anomalous.

In some embodiments, information systems located inside and outside of a medical facility can access medical data of the medical facility as long as authentication is successfully performed (e.g., using a security credential). For example, a login/password combination may be used for authentication, although other types of authentication protocols may be used as well. Referring to FIG. 7, medical data repository 420 manages distribution of data to information system 700 (which may be any of information systems 112a, 112b, 310, for example). At block 710, information system 710 initiates a query/retrieval request via a secure channel such as a virtual private network (VPN) tunnel or a HTTPS channel following an SSL handshake. At block 720, medical data repository 420 checks the registration status of information system 700. For example, medical data repository 420 may have a registry (e.g., list stored in memory) of registered information systems, and medical data repository 420 may verify whether information system 700 is in the registry. If the registration status of information system 700 is confirmed, medical data repository 730 authenticates information system 730, e.g., using an authentication protocol.

In some embodiments, an information distribution management mechanism of medical data repository 420 sets and enforces different privileges for respective information systems, which may be located inside or outside of a medical facility. The querying and retrieval of data may be controlled by parameters pertaining to these privileges, and the parameters may be stored inside data repository 420. Parameters pertaining to privileges may include allowable data types (e.g., blood pressure allowed; oxygen saturation level disallowed), allowable time range of data (e.g., data only from Jan. 5, 2014 between 1:00 pm and 5:00 pm), allowable interval of data (e.g., data every 30 minutes), allowable location of data (e.g., only data from a particular facility), allowable frequency of query and retrieval (e.g., a particular information system can only query and retrieve data 5 times per day) and others. The information distribution management mechanism may support different business models where, for example, privileges are maintained on a per-information-system basis and fees are assessed to information systems on the basis of such privileges or on the basis of actual data consumption. At block 740, the information distribution management mechanism evaluates the received query/retrieval request based on the privileges corresponding to information system 700 stored at medical data repository 420. At block 750, medical data repository 420 responds to the query/retrieval request based on the stored privileges of information system 700.

Similar to the registration check and authentication performed by medical data repository 730 for communications with information system 700, a registration check and an authentication may be performed by medical data repository 730 at the beginning of any communication with data processor 410. Registration info associated with registered data processors may be stored at data repository 730.

In some embodiments, medical data repository sends to a medical information system (e.g., information system 112a, 112b, or 310) a summary statistic associated with at least a portion of monitored medical data or corresponding metadata, instead of sending the raw medical data or metadata itself. By sending a summary statistic (e.g., mean, standard deviation, median, first occurrence, last occurrence, weighted scaled value, any predetermined quantile or percentile, etc.) of the data that may be calculated for a requested time interval, the amount of data transmitted on local network 110a and/or other networks (e.g., networks 110b, 110c) can be reduced, the processing load for information systems can be reduced, and information systems that require relatively little data can be accommodated.

Because of the ability to transport data according to a set of privileges to information systems inside and outside of facilities, various embodiments also enable data related to medical equipment (e.g., medical equipment usage, medical equipment configuration, and other data) to be transported to medical equipment manufacturers. For example, the data related to medical equipment may first be stored at data repository 420 and then made available to medical equipment manufacturers. Such data related to medical equipment provides medical equipment manufacturers with insight as to, e.g., how users use their equipment and which features of the equipment are used more frequently. Such data related to medical equipment is valuable for product development and product marketing.

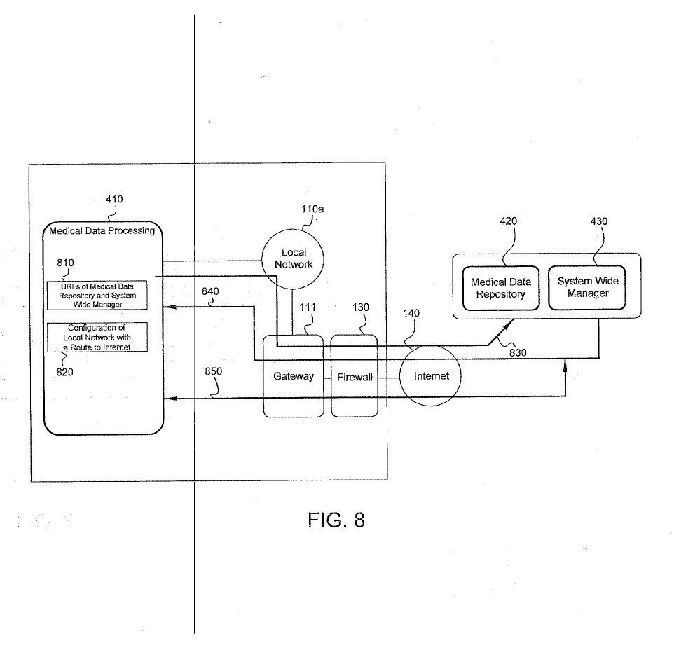

Referring to FIG. 8, data processor 810 stores URLs 810 of data repository 420 and system-wide manager 430 (block 810) and configuration data 820 regarding local network 110a having a route to the Internet 140. A secured communication between medical data processor 410 and medical data repository 420 and between medical data processor 410 and system-wide manager 430 may, in some embodiments, be always initiated by data processor 410, as shown by arrow 830. The communication may be initiated using a predetermined port, each a port reserved for email or Internet traffic, because such ports are typically approved for outbound traffic by corporate firewalls. For example, TCP ports 80 or 443 reserved for HTTP and HTTPS, respectively, may be used, or TCP ports 25 or 465 reserved for email may be used. The initiation of communication may be performed regularly (e.g., hourly, daily, weekly, etc.). Data processor 410 may initiate communication to internally pre-programmed URLs of data repository 420 and system-wide manager 430. These URLs may be registered to the Internet domain registrar so that the traffic from data processor 410 will route correctly to devices corresponding to those URLs. While there is a possibility that a rogue router could attempt to intercept a communication and route to a different destination, even this scenario is handled by various embodiments because data processor 410 is expected to initiate communication regularly, enabling system-wide manager 430 to detect any abnormality with respect to a predetermined temporal pattern (e.g., absence of an expected “heartbeat” communication from data processor 410) and create an alert accordingly. Data repository 420 and system-wide manager 430 may communicate between themselves, e.g., to notify one another of communications initiated by data processor 410. The communication initiation (by data processor 410) and acceptance (by data repository 420 and system-wide manager 430) may themselves be secured and together establish a secure communication using modern encryption or other type of secure communication protocol.

As shown by arrow 840, medical data repository 420 and/or system-wide manager 430 respond to the initiation of the secure connection by establishing a secure channel, e.g., through a reserved Internet port or email port.

In some embodiments, a secure technique is provided to perform automatic remote update/upgrade on the deployed data processors 410 via system-wide manager 430 without any physical, at-location interaction needed on the part of any human (e.g., facility's IT staff). In this way, security vulnerabilities associated with Remote Desktop and similar programs are eliminated. As shown by arrow 850, data processor 410 sends to system-wide manager 430 a request for a software update, e.g., one or more specific software updates or a list of any available software updates. Data processor 410 receives the software update(s) from system-wide manager 430 and automatically performs the software update(s) at data processor 410 without human intervention. The remote update technique allows data processor 410 to revert back to the last known working configuration if/when an update fails.

The communication between data processor 410 and data repository 420 and the communication between data processor 410 and system-wide manager 430 may use a secured method wherein communication is initiated on an as-needed basis by data processor 410, unlike traditional approaches to Remote Desktop-based maintenance where a server would need to be run to open a port to listen for any inbound request at any time. Thus, by changing the directionality and eliminating the always-listening port functionality, a security vulnerability associated with traditional port-based Remote Desktop is avoided.

In addition to remote upgrade, system-wide manager 430 may also use the same secured technique to provide system-wide monitoring of each deployed data processor 410. Each data processor 410 may regularly (e.g., on a periodic basis such as daily, hourly, or at some other regular interval) send component status information, including status of the medical equipment 102, and/or of the local network 110a and/or of that data processor 410, to system-wide manager 430. System-wide manager 430 may check the registration status and authentication status of data processor 410 for each such data communication. Based on such data received from data processor 410, system-wide manager 430 can alert the facility's IT staff to issues related to any data processor 410. The facility's IT staff can then correct those issues before they become major problems. System-wide manager 430 may also report usage and operational statistics to relevant users and be able to generate customized reports for the component status information.

System-wide manager 430 may be configured to access data sent by data processor 410, e.g., data such as status and/or configuration of the medical equipment, operational integrity data of the network (e.g., WiFi outage data), data regarding status of the data processor 410 and other data. In some embodiments this accessed information can be received and stored in system-wide manager 430, and in other embodiments this accessed data is stored in medical data repository 420. One or more databases for storing this accessed information can be located at data repository 420, at system-wide manager 430, or distributed between the two. System-wide manager 430 may have its own algorithm to detect any anomaly. For example, in some embodiments system-wide manager 430 can detect missing data from data processor 410 within a predetermined time frame. System-wide manager 430 may present relevant data to relevant users and can generate an alert to relevant users. The alert can be programmable to different levels and directed to different relevant users. System-wide manager 430 may also be configured to perform and report data analyses on usage and operational information related to the medical equipment 102, local network 110a and itself (system-wide manager 430) to relevant users. System-wide manager also provides software updates for which data processor 410 can initiate retrievals.

Thus, by introducing medical data repository 420 as an intermediary in the data chain between data processor 410 and information systems (e.g., 112a, 112b, 310), several advantages are achieved relative to traditional medical data connectivity approaches. Data processor 410 does not have to be responsible for disseminating data to various information systems, which might have differing requirements regarding the type or granularity of data. Data processor 410 does not need to keep track of network information for various networks, instead only maintaining network configuration data of local network 110a. Offloading to data repository 420 the task of storing and disseminating the data reduces the burden on data processor 104. Because data repository 420 is moved outside the medical facility containing local networks 110a, 110b, and 110c, the stored data can be accessed by any entity easily as long as that entity is registered and can be authenticated. By presenting a unified interface to various medical monitoring devices 102, data processors can finely control the data acquired from monitoring devices, e.g., by increasing or decreasing frequency of measurements based on various conditions.

Additionally, system-wide manager 430 provides other advantages over traditional medical data processing approaches. Monitoring of various equipment, data processor conditions, and network conditions is provided, and detection of missed heartbeats can provide early indication of anomalies. Security is enhanced through the elimination of open ports that are otherwise needed for remote upgrade in traditional approaches. Data is made available to entities that need it, in the format desired, and based on privileges of individual information systems.

These and other advantages accrue to the benefit of patients (who may receive improved patient care, e.g., through timely increases in temporal frequency of monitored characteristics), hospitals, clinics, IT personnel, downstream data consumers, medical equipment manufacturers, and others.

Data repository 420 and/or system-wide manager 430 can each be implemented by a general purpose computer programmed in accordance with the principles discussed herein. It may be emphasized that the above-described embodiments, particularly any “preferred” embodiments, are merely possible examples of implementations, merely set forth for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a computer readable medium. The computer readable medium can be a machine-readable storage device, a machine-readable storage substrate, a memory device, or a combination of one or more of them.

The term “processor” encompasses all apparatus, devices, and machines for processing data, including by way of example a programmable processor, a computer, or multiple processors or computers. The processor can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more data memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, to name just a few.

Computer readable media suitable for storing computer program instructions and data include all forms data memory including non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

Computing systems in accordance with various embodiments can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

FIG. 9 is an architecture diagram of a computer 900 that may be used in some embodiments, and one or more components of computer 900 can be used for implementing system-wide manager 420 and/or data repository 430. Computer system 900 may include one or more processors 902. Each processor 902 is connected to a communication infrastructure 906 (e.g., a communications bus, cross-over bar, or network). Computer system 900 may include a display interface 922 that forwards graphics, text, and other data from the communication infrastructure 906 (or from a frame buffer, not shown) for display on the display unit 924.

Computer system 900 may also include a main memory 904, such as a random access memory (RAM), and a secondary memory 908. The secondary memory 908 may include, for example, a hard disk drive (HDD) 910 and/or removable storage drive 912, which may represent a floppy disk drive, a magnetic tape drive, an optical disk drive, a memory stick, or the like as is known in the art. The removable storage drive 912 reads from and/or writes to a removable storage unit 916. Removable storage unit 916 may be a floppy disk, magnetic tape, optical disk, or the like. As will be understood, the removable storage unit 916 may include a computer readable storage medium having tangibly stored therein (embodied thereon) data and/or computer software instructions, e.g., for causing the processor(s) to perform various operations. Main memory 904 or secondary memory 908 may be used for implementing the data repository.

In alternative embodiments, secondary memory 908 may include other similar devices for allowing computer programs or other instructions to be loaded into computer system 900. Secondary memory 908 may include a removable storage unit 918 and a corresponding removable storage interface 914, which may be similar to removable storage drive 912, with its own removable storage unit 916. Examples of such removable storage units include, but are not limited to, USB or flash drives, which allow software and data to be transferred from the removable storage unit 916, 918 to computer system 900.

Computer system 900 may also include a communications interface (e.g., networking interface) 920. Communications interface 920 allows software and data to be transferred between computer system 900 and external devices. Examples of communications interface 920 may include a modem, Ethernet card, wireless network card, a Personal Computer Memory Card International Association (PCMCLA) slot and card, or the like. Software and data transferred via communications interface 920 may be in the form of signals, which may be electronic, electromagnetic, optical, or the like that are capable of being received by communications interface 920. These signals may be provided to communications interface 920 via a communications path (e.g., channel), which may be implemented using wire, cable, fiber optics, a telephone line, a cellular link, a radio frequency (RF) link and other communication channels.

It is understood by those familiar with the art that various embodiments may be implemented in hardware, firmware, or software encoded on a non-transitory computer-readable storage medium.

The systems and processes are not limited to the specific embodiments described herein. In addition, components of each system and each process can be practiced independent and separate from other components and processes described herein.

The previous description of the embodiments is provided to enable any person skilled in the art to practice the disclosure. The various modifications to these embodiments will be readily apparent to those skilled in the art, and the generic principles defined herein may be applied to other embodiments without the use of inventive faculty. Thus, the present disclosure is not intended to be limited to the embodiments shown herein, but is to be accorded the widest scope consistent with the principles and novel features disclosed herein.

What is claimed is:

1. A system comprising:
a first computer located in a patient care facility, the first computer including:
a first processor;
a memory;
a first communications interface connecting the first computer to one or more medical monitoring devices configured to monitor a plurality of characteristics of a patient in the patient care facility;
a second communications interface connecting the first computer to a first local area network;
a non-transitory computer readable storage medium tangibly embodying first instructions executable to cause the first processor to:
receive first data from the one or more medical monitoring devices, the first data representing the plurality of monitored characteristics of the patient or metadata associated with the plurality of monitored characteristics;
send the first data via the second communications interface to a predetermined IP address, wherein the predetermined IP address is stored in the memory of the first computer;
detect that a value of one of the monitored characteristics is within a predetermined range; and
set a polling frequency for each of the monitored characteristics to a common frequency based on said value being within the predetermined range;
a set of one or more computers outside the first local area network, the set of one or more computers including:
a second processor;
a database;
a third communications interface connecting each computer in the set to a public network, wherein the first local area network is connected to the public network; and
a non-transitory computer readable storage medium tangibly embodying second instructions executable to cause the second processor to:
receive the first data via the third communications interface;
store the first data in the database;
receive a data request from a medical information system via the public network, wherein the medical information system is registered with one of the computers in the set of one or more computers;
verify a registration status of the medical information system;
authenticate the medical information system; and
only if the registration status is successfully verified and the medical information system is successfully authenticated, send second data to the medical information system, the second data representing at least one monitored characteristic of the patient, usage data associated with the medical monitoring device, or status data associated with the medical monitoring device, or a combination thereof.

2. The system of claim 1, wherein:
the first instructions are further executable to cause the first processor to:
receive, via the first communications interface, third data representing usage data associated with at least one of the medical monitoring devices; and
send, via the second communications interface, the third data to one of the computers in the set of one or more computers; and
the second instructions are further executable to cause the second processor to:
receive the third data via the third communications interface; and
store the third data in the database.

3. The system of claim 1, wherein:
the first instructions are further executable to cause the first processor to:
receive, via the first communications interface, third data representing an operational status of at least one of the medical monitoring devices; and
send, via the second communications interface, the third data to one of the computers in the set of one or more computers; and the second instructions are further executable to cause the second processor to:
receive the third data via the third communications interface; and
store the third data in the database.

4. The system of claim 1, wherein:
the first instructions are further executable to cause the first processor to:
send, via the second communications interface, third data representing a status of the first computer; and
the second instructions are further executable to cause the second processor to:
receive the third data via the third communications interface; and
store the third data in the database.

5. The system of claim 1, wherein:
the first instructions are further executable to cause the first processor to:
send, via the second communications interface, third data representing a status of the first local area network; and
the second instructions are further executable to cause the second processor to:
receive the third data via the third communications interface; and
store the third data in the database.

6. The system of claim 1, wherein the first instructions are further executable to cause the first processor to:
detect an anomalous connection between one of the medical monitoring devices and the first computer; and
send to one of the computers in the set of one or more computers, via the second communications interface, a notification of the anomalous connection.

7. The system of claim 6, wherein the first communications interface includes a wired interface, and the first instructions are further executable to cause the first processor to detect the anomalous connection by testing the wired interface.

8. The system of claim 7, wherein the first instructions are further executable to cause the first processor to detect the anomalous connection by:
sending a request for data to said medical monitoring device;
checking for a response to the request from said medical monitoring device;
determining the anomalous connection based on a lack of response or a faulty response from said medical monitoring device.

9. The system of claim 1, wherein the first computer is registered with one of the computers in the set of one or more computers, and the second instructions are further executable to cause the second processor to verify a registration status of the first computer

10. The system of claim 9, wherein second instructions are further executable to cause the second processor to:
if the first computer is verified as a registered computer, perform an authentication check on the first computer.

11. The system of claim 9, wherein the first instructions are further executable to cause the first processor to:
initiate a communication with one of the computers in the set of one or more computers using a predetermined port.

12. The system of claim 1, wherein the first instructions are further executable to cause the first processor to:
send to one of the computers in the set of one or more computers, via the second communications interface, a request for a software update;
receive the software update from said one computer in the set of one or more computers; and
automatically perform the software update at the first computer without human intervention.

13. The system of claim 1, wherein the first instructions are further executable to cause the first processor to initiate communications with one of the computers in the set of one or more computers according to a predetermined temporal pattern, and the second instructions are further executable to cause the second processor to:
detect an irregularity in communications from the first computer with respect to the predetermined temporal pattern, and
send an alert based on the detected irregularity.

14. The system of claim 1, wherein the second instructions are further executable to cause the second processor to send to the medical information system a summary statistic associated with at least a portion of the first data.

15. The system of claim 1, wherein the first instructions are further executable to cause the first processor to:
detect that a value of one of the monitored characteristics is outside a predetermined range; and
decrease a polling frequency for at least one of the monitored characteristics based on the detected value of one of the monitored characteristics being outside the predetermined range.

16. A method comprising:
at a first computer connected to one or more medical monitoring devices, receiving first data from one of the medical monitoring devices repetitively according to a programmable time interval, wherein the first computer is within a first local area network, the one or more medical monitoring devices are configured to monitor a plurality of characteristics of a patient in a patient care facility, and the first data represent one or more monitored characteristics of the patient or metadata associated with said one or more monitored characteristics,
sending the first data from the first computer to a second computer via the local area network and a public network, wherein the second computer is outside the first local area network and is connected to the public network, and the first data is sent from the first computer to the second computer based on an IP address of the second computer stored in a memory of the first computer;
determining that a value of one of the monitored characteristics is within a predetermined range; and
setting a polling frequency for each of the monitored characteristics to a common frequency based on said value being within the predetermined range.

17. The method of claim 16, further comprising:
at the first computer, receiving second data representing usage data associated with the medical monitoring device or status data associated with the medical monitoring device; and
sending the second data to the second computer via the local area network and the public network.

18. The method of claim 16, further comprising:
send second data from the first computer to the second computer via the local area network and the public network, the second data representing a status of the first computer or of the first local area network.

19. The method of claim 16, further comprising:
detecting an anomalous connection between the medical monitoring device and the first computer; and
sending to the second computer a notification of the anomalous connection.

20. A method comprising:
receiving first data sent from a first computer connected to a medical monitoring device, wherein the first computer is within a first local area network, the medical monitoring device is configured to monitor at least one characteristic of a patient in a patient care facility, the first data represent one or more monitored characteristics of a patient or metadata associated with said one or more monitored characteristics, and the first data are received at a second computer outside the first local area network,
storing the first data in a database;
receiving a communication request from a medical information system via a second local area network and the public network, wherein the medical information system is on the second local area network and is registered with the second computer;
verifying a registration status of the medical information system;
authenticating the medical information system using an authentication protocol; and
if the registration status is successfully verified and the medical information system is successfully authenticated, sending a portion of the first data to the medical information system via the public network and the second local area network.

21. The method of claim 20, further comprising:
receiving, from the first computer, second data including usage data associated with the medical monitoring device, status data associated with the medical monitoring device, or a combination thereof;
storing the second data in the database; and
if the registration status is successfully verified and the medical information system is successfully authenticated, sending a portion of the second data to the medical information system via the public network and the second local area network.

22. The method of claim 20, further comprising:
maintaining, at a memory of the second computer, a registry of registered computers;
verifying whether the first computer is one of the registered computers; and
if the first computer is one of the registered computers, authenticating the first computer.
